(19) United States
(12) Plant Patent Application Publication
(10) Pub. No.: US 2012/0264195 P1
(43) Pub. Date: Oct. 18, 2012

(54) High bio-productivity chlamydomonas strain DG8-108

(50) ;

(76) Inventors: Karl Y. Biel, Tucson, AZ (US); Vladimir G. Ladygin, Pushchino (Moscow Region) (RU); Calvin M. Nii, Boulder, CO (US)

(21) Appl. No.: 13/066,575

(22) Filed: Apr. 18, 2011

(51) Int. Cl. C12N 1/12

(52) U.S. Cl. 435/257.6

(57) ABSTRACT

A novel strain of the unicellular green algae Chlamydomonas reinhardtii, named “Strain DG8-108”, was created by exposing wild-type C. reinhardtii to γ-radiation exposure. This new strain demonstrates nearly double the biomass and photosynthetic productivity of wild-type C. reinhardtii under both optimal and stress conditions.

### LATIN NAME OF GENUS AND SPECIES

Chlamydomonas reinhardtii (Dang) DG8-108

### I. BACKGROUND OF THE INVENTION

This application describes a novel phototrophic strain of Chlamydomonas reinhardtii, known as strain DG8-108, that can accumulate 2.5 times more biomass on mineral medium and under photosynthesis, compared to wild-type, under optimal and stressful environmental conditions. This novel strain was developed for use in plant physiological research and in the commercial production of bioproducts.

Chlamydomonas is a genus of unicellular green algae (Chlorophyta). These algae are found in soil, fresh water, oceans, and even in snow on mountaintops. Algae in this genus have a cell wall, a chloroplast, an “eyespot” that perceives light, and two anterior flagella with which they can swim using a breast-stroke type motion. More than 500 different species of Chlamydomonas have been described, but most scientists work with only a few species.

The most widely used laboratory species is Chlamydomonas reinhardtii (Dang). The wild-type of this species (strain 137C) was isolated from soil by Dr. Smith in 1948 in USA (see in rf. Levine 1960). Cells of this wild-type strain are haploid, and can grow on a simple medium of inorganic salts, using photosynthesis to provide energy. Cells can also grow in total darkness when acetate is provided as an alternative carbon source. When deprived of nitrogen, haploid cells of opposite mating types can fuse to form a diploid zygospore which forms a hard outer wall that protects it from adverse environmental conditions. When conditions improve (e.g. when nitrogen is restored to the culture medium), the diploid zygote undergoes meiosis and releases four haploid cells that resume the vegetative life cycle.

We used C. reinhardtii strain 137C, which was originally sent to us by Dr. R. P. Levine in 1965, to create the C. reinhardtii strain DG8-108 described herein to facilitate investigation of general plant physiological phenomena.

### II. SUMMARY OF THE INVENTION

The following traits distinguish C. reinhardtii strain DG8-108 from wild-type and other laboratory-created (mutant) C. reinhardtii strains known to the inventors:

1) Strain DG-8-108 has higher photosynthetic rates—2.5 times higher—than wild-type C. reinhardtii;

2) Strain DG-8-108 has higher overall total productivity—2 times higher—compared to wild-type C. reinhardtii;

3) Strain DG-8-108 has the ability to accumulate 2 to 2.5 higher biomass levels compared to wild-type C. reinhardtii; and,

4) Strain DG-8-108 has the ability to maintain these enhanced physiological attributes under both optimal and stressful environmental conditions.

### III. DESCRIPTION OF DRAWINGS

FIG. 1. Scanning electron micrographs of the novel C. reinhardtii strain DG8-108. Ultrastructure of the whole cell (1) and close-ups of the chloroplast membranes in novel strain DG8-108 (A) and wild-type (B) of Strain DG8-108.

FIG. 2. Productivity of wild-type Chlamydomonas reinhardtii and Strain DG-8-108. Triangle—Strain DG-8-108; circle—wild-type.

FIG. 3. Pigment content in wild-type Chlamydomonas reinhardtii and Strain DG-8-108. Dark—Strain DG-8-108; white—wild-type.

FIG. 4. Pigment content during stress treatment in wild-type Chlamydomonas reinhardtii and Strain DG-8-108. Triangle—DG8-108; circle—wild-type.

FIG. 5. Stress tolerance of wild-type Chlamydomonas reinhardtii and Strain DG-8-108 to complex stress conditions including: low light intensity (<42 μmol photons/m² s), low carbon dioxide level (<0.3%), low levels of nutritional compounds, and exposure to bacterial (Escherichia coli), coccus (Staphylococcus spp.) and fungal infection (Penicillum spp.), etc. Triangle—Strain DG-8-108; circle—wild-type.

### IV. DETAILED METHODS, MORPHOLOGICAL AND PHYSIOLOGICAL DESCRIPTION

### Method

The novel strain DG8-108 was created by exposing wild-type C. reinhardtii strain 137-C to γ-radiation. The wild-type cells 137C(+) from agar (agar-Difco) plates were transferred into liquid mineral medium (without acetate) in sterile box, and illuminated 10-12 hrs before finishing one cell division.

This suspension of young cells, at a density 1 million cells per 1 ml of medium, was then treated with gamma-radiation at a dose 200 grays. Cobalt-60 was used as the γ-radiation source (60 grays from the top). At this dose, about 1% of the total cells survived. A suspension of surviving cells was then transferred into surface of agar-medium in Petri dishes.

The density of grown cells was not/should not be more than 200 colonies per Petri-dish. After 10-14 days growth, the surviving cells formed colonies of 3-5 mm in diameter. The dark-green colonies were separated, collected and, then analyzed for various morphological and physiological properties. The strain demonstrating the highest productivity among all the mutant strains analyzed was a dark-green mutant that was subsequently named: strain DG8-108.

### Morphological and Physiological Characteristics

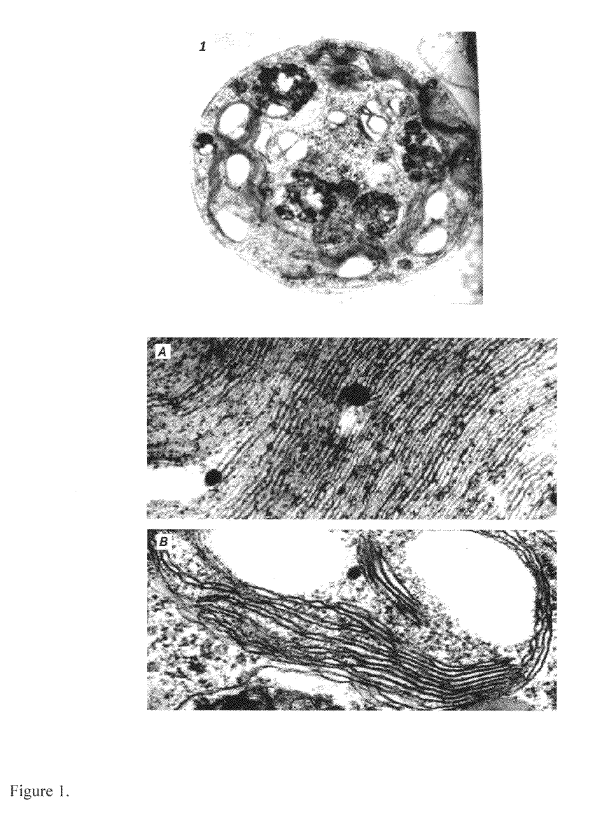

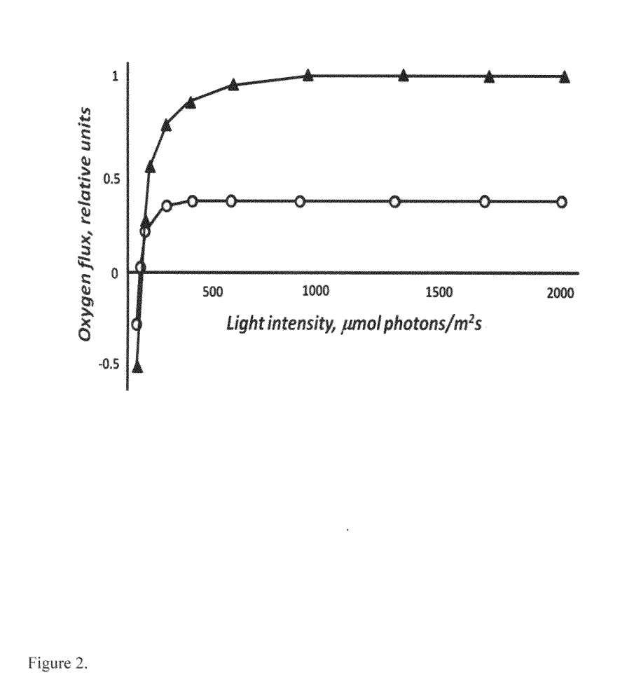

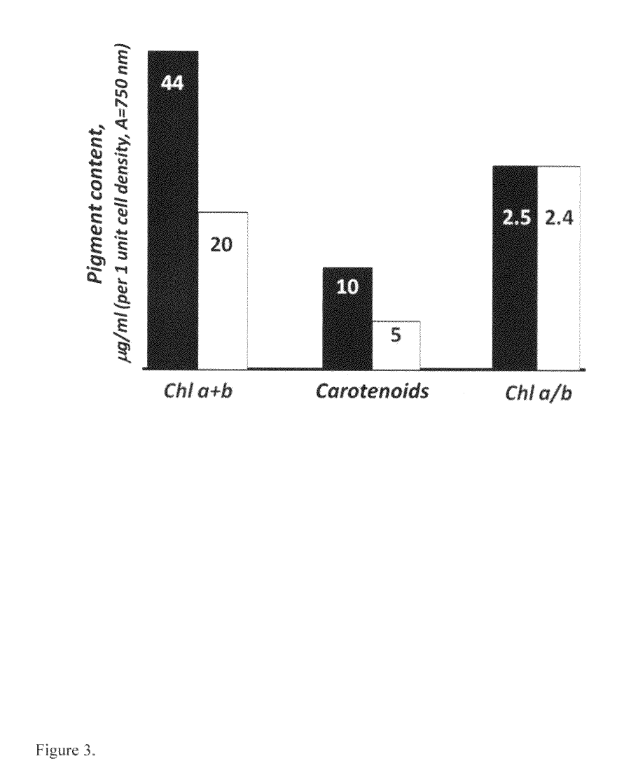

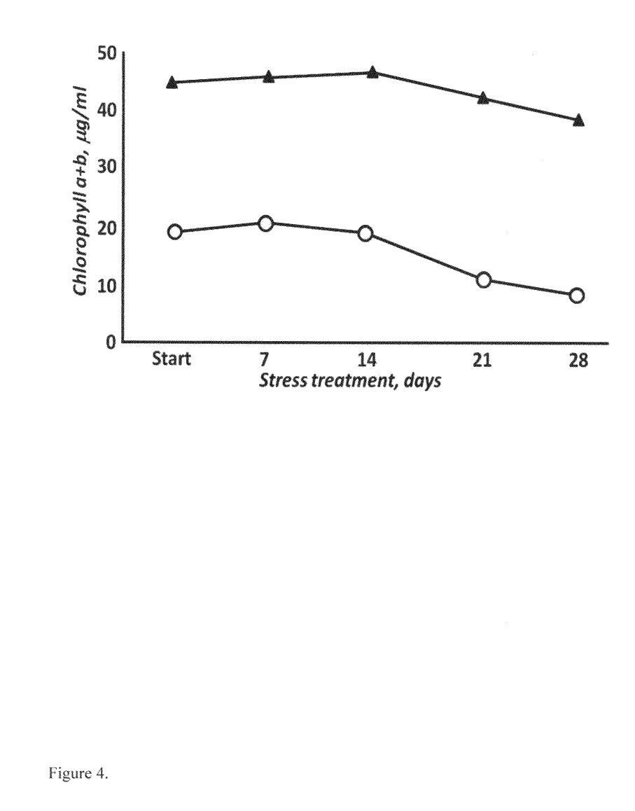

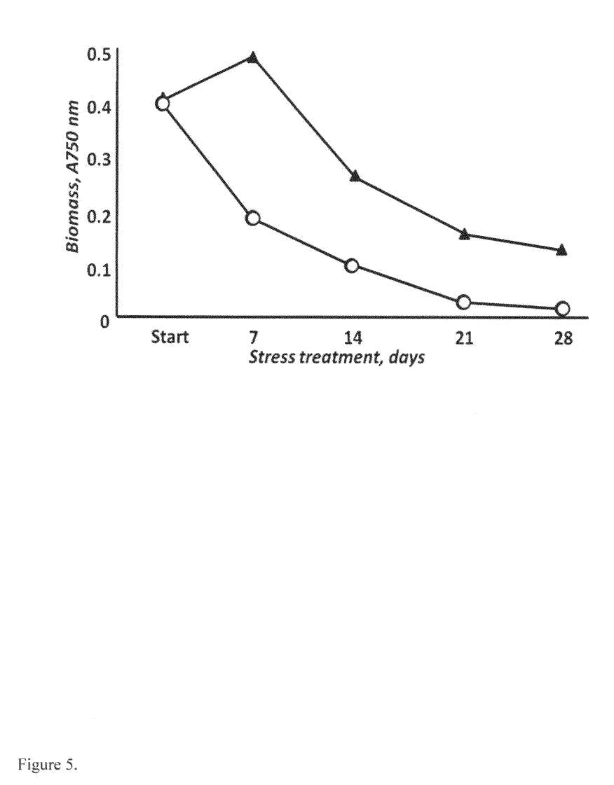

The novel C. reinhardtii strain DG8-108 has the following characteristics:
Strain DG8-108 cells may be range from 18-25 microns (wild-type—10-15 microns) in length and have either a spherical and/or ovate morphology.
Strain DG8-108, when grown on agar (agar-Difco) plates with mineral medium (18-20 g/L agar), form dark green colonies that are 0.3-0.5 cm in diameter within 10 days. The size of mutant colonies in 1.3-1.8 time greater than size of wild type colonies.
Strain DG8-108 grown in liquid media (made with either distilled or tap water because pH of medium is maintained using buffer) with 0.2% sodium acetate achieves cell densities of 40 million cells per milliliter within 48 hours. The mutant grown in mineral medium grows 1.5 times slower than in medium containing 0.2% acetate.
Both solid and liquid “mineral media” contained the following compounds (mg/L): NH₄CL (0.4); MgSO₄.7H₂O (0.1); CaCl₂.2H₂O (0.05); K₂HPO₄ (0.75); and KH₂PO₄ (0.36). In addition, a “microelement solution” was added to media at a concentration of 1 mg/L. This “microelement solution” contained the following compounds (mg/L): EDTA, sodium salt (50.00); ZnSO₄.7H₂O (22.00); H₃BO₃ (11.40); MnCl₂.4H₂O (5.06); FeSO₄. 7H₂O (4.99); CaCl₂.6H₂O (1.61); CuSO₄.5H₂O (1.57); and (NH₄)₆Mo₇O₂₄.4H₂O (1.10). To prevent precipitation, 0.5 grams per liter tri-sodium citric acid should be included in the microelement solution.
The optimal temperature for Strain DG8-108 grown on agar medium is 23-25° C.; for mutants grown in liquid medium the optimal temperature is 27-35° C.
In natural conditions, Strain DG8-108 may be cultivated in a very wide temperature range between 20° C. to 37° C.
The optimal light intensity for Strain DG8-108 grown on agar is 42-56 gmol photons/m² s; for Strain DG8-108 grown in liquid medium, the optimal light intensity is 182-210 μmol photons/m² s. The illumination for both conditions was delivered by mercury luminescence lamps (LB-40, white light).
The optimal pH of the growth medium is 6.8-7.2. Under culture using solid media (agar), the pH of the solid media may increase from 6.8 to 7.2 over a 14-day period under constant light conditions; in liquid medium, a similar pH change may occur in 4 days.
Strain DG8-108 grown in liquid medium achieve a total biomass of 5 to 10 grams fresh weight per liter after 3 days. The maximum total biomass achieved in 3 days is 20 grams fresh weight per liter.
Strain DG8-108 growth rate in culture is not affected by seasonal variation.
Strain DG8-10's growth rate may be increased by increasing the concentration of medium salts by 5 times compared to normal growth medium. Its doubling time can be decreased from 10-12 hours to 6 hours—and therefore, biomass levels will nearly double—due to increased salt levels.
Adult cells of Strain DG8-108 have a very large chloroplast that occupies nearly ⅔ of the total cell volume, and the chloroplast has 1.9-2.0 (SD =0.4) times more thylakoid membranes compared to wild-type (FIG. 1. Number of thylakoids calculated on basis of chloroplast cross section surface. Magnification was 65,000×).
The total productivity of Strain DG8-108, on both a gross and net basis is, at minimum, 2-2.5 times greater than wild-type (FIG. 2).
The chlorophyll content (chl a+chl b) of Strain DG8-108 is nearly double that of the wild-type (FIG. 3) and persists even under chronic stress conditions (FIG. 4).
Strain DG8-108 shows increased stress tolerance (higher pigment content and higher survivorship demonstrated by Strain DG8-108 (compared to wild-type) to extreme environmental conditions (FIGS. 4 and 5).

### V. LITERATURE CITED

1. Levine RP 1960 Genetic control of photosynthesis in Chlamydomonas reinhardtii . Proc. Nat. Acad. Sci. U.S.A., 16, 972.

What is claimed is:

1. A new and distinct variety of alga of the genus Chlamydomonas, named Chlamydomonas reinhardtii strain DG8-108, substantially as herein shown and described, characterized by the fact that this alga demonstrated 2.5 times higher rates of photosynthesis, 2 times greater total productivity, 2 times greater biomass accumulation and higher environmental stress tolerance compared to wild-type.
